(19) United States
(12) Patent Application Publication
(10) Pub. No.: US 2026/0325567 A1
(43) Pub. Date: Oct. 22, 2026

(54) Increasing Sensitivity of Liquid Biopsies with Pre-Application of a DNASE1L3 Inhibitor Agent

(71) Applicant: BillionToOne, Inc., Menlo Park, CA (US)

(72) Inventor: David Tsao, San Carlos, CA (US)

(73) Assignee: BillionToOne, Inc., Menlo Park, CA (US)

(21) Appl. No.: 19/653,818

(22) Filed: Apr. 21, 2026

Related U.S. Application Data
(60) Provisional application No. 63/792,066, filed on Apr. 21, 2025.

(51) Int. Cl. A61K 31/727; A61B 5/15; A61K 31/616; A61K 31/721; C12Q 1/6883; C12Q 1/6886

(52) U.S. Cl. CPC A61K 31/727; A61B 5/150007; A61K 31/616; A61K 31/721; C12Q 1/6883; C12Q 1/6886; C12Q 2600/156

(57) ABSTRACT

The subject matter described relates generally to liquid biopsies and, in particular, to an approach to improving sensitivity of such biopsies by administering a DNASE1L3 inhibitor agent to the subject prior to conducting a blood draw.

### CROSS REFERENCE TO RELATED APPLICATIONS

The present application claims the benefit of U.S. Provisional Patent Application No. 63/792,066 filed Apr. 21, 2025 which is incorporated herein by reference in its entirety.

### TECHNICAL FIELD

The subject matter described relates generally to liquid biopsies and, in particular, to an approach to improving sensitivity of such biopsies by administering a DNASE1L3 inhibitor agent to the subject prior to conducting a blood draw.

### BACKGROUND INFORMATION

Cell-free DNA (cfDNA) diagnostics have revolutionized non-invasive prenatal testing (NIPT) and are increasingly applied in cancer detection through liquid biopsy approaches. These minimally invasive techniques have demonstrated significant clinical utility by enabling the detection of genetic abnormalities in fetuses and genomic alterations in tumors through a simple and convenient blood draw, without the risks associated with tissue biopsies.

A fundamental challenge limiting the broader application of cfDNA diagnostics is the extremely low concentration of cfDNA in peripheral blood. On average, only approximately 1000 haploid genome equivalents per mL of blood plasma are present, making reliable detection challenging. This limitation is particularly pronounced in cancer liquid biopsy applications, where circulating tumor DNA (ctDNA) typically represents 0.1% or less of the total cfDNA in cancer patients, often translating to just a few detectable molecules per milliliter of plasma.

### SUMMARY

The above and other problems may be addressed by priming a subject before a liquid biopsy blood draw with the application of a DNASE1L3 inhibitor agent (e.g., heparin, aspirin, etc.). In various embodiments, a subject is first administered a dose of the agent and then a predetermined time later (e.g., approximately one hour, approximately two to three hours, or approximately twelve hours later), a blood draw is conducted to obtain a blood sample. An assay is performed on the blood sample that includes extracting cfDNA, forming a sequencing library using the cfDNA, performing sequencing on the sequencing library to generate sequencing results, and analyzing the sequencing results to make a diagnostic determination. The diagnostic determination may be, for example, determining the presence or absence of a genetic condition in a fetus, detecting the presence of a tumor in a cancer patient, or calculating one or more metrics indicating disease progression in a cancer patient.

In one embodiment, a clinical assay provider recommends the application of an agent to a subject prior to a blood draw. The agent decreases the fetal or tumor fraction while increasing the total amount of cfDNA in the subject's blood such that the total number of fetal-originated or tumor-originated molecules is increased relative to if the subject had not been administered the agent. For example, the agent may be a blood thinner or other DNASE1L3 inhibitor such as heparin or aspirin. The clinical assay provider receives a blood sample obtained by the blood draw and analyzes the blood sample to make the clinical determination.

In another embodiment, a clinical assay provider recommends the application of heparin a subject a predetermined amount of time prior to a blood draw. The clinical assay provider receives a blood sample obtained by the blood draw and analyzes the blood sample to make the clinical determination.

In some embodiments, the clinical assay provider instructs the subject, a physician, or another clinician to administer the agent. For example, the subject may self-administer the agent the night before or morning of a scheduled blood draw. A physician or other clinician may conduct the blood draw at approximately a time relative to the application of the agent that was specified by the clinical assay provider. The blood sample obtained by the blood draw may then be sent to the clinical assay provider (e.g., to a testing laboratory) for analysis. Alternatively, the clinical assay provider may conduct the blood draw and perform the analysis, in which case, the blood sample is received by the testing equipment of the clinical assay provider.

### BRIEF DESCRIPTION OF THE DRAWINGS

FIG. 1 illustrates an increase in cfDNA in a patient's blood after beginning heparin treatment.

FIG. 2 illustrates an increase in circulating tumor DNA in the blood of the patient from FIG. 1 after beginning heparin treatment.

FIG. 3 is a flowchart illustrating an example diagnostic workflow for administering heparin prior to conducting a blood draw for an assay, according to one embodiment.

FIGS. 4A and 4B illustrate an increase in cfDNA isolated from subjects after heparin exposure compared to cfDNA collected prior to heparin exposure.

FIG. 5 illustrates the increase in total amount of cfDNA from vascular endothelial cells by exposure to heparin.

### DETAILED DESCRIPTION

The figures and the following description describe certain embodiments by way of illustration only. One skilled in the art will readily recognize from the following description that alternative embodiments of the structures and methods may be employed without departing from the principles described. Wherever practicable, similar or like reference numbers are used in the figures to indicate similar or like functionality. Where elements share a common numeral followed by a different letter, this indicates the elements are similar or identical. A reference to the numeral alone generally refers to any one or any combination of such elements, unless the context indicates otherwise.

Blood Thinners Increase Total cfDNA

Patients treated with blood thinners such as heparin or heparin derivatives (e.g. enoxaparin) have been reported to be associated with 20-fold elevated failure rate for NIPT. While failure rates for NIPT are driven by low fetal fraction, heparin is also associated with massively increased levels of total cfDNA. These facts appear to be contradictory on their face. However, evaluation of available data indicates that while fetal fraction decreases with the application of heparin, the total amount of fetus-originated molecules in the mother's blood is higher than the total number of fetus-originated molecules for patients who have not taken heparin. This is because the increase in total cfDNA caused by heparin is greater than the reduction in the number of fetus-originated molecules caused by the reduction in fetal fraction. Put another way, heparin increases the number of fetus-originated molecules in the mother's blood, but by a lesser amount than heparin increases the amount of cfDNA from other sources.

This observation was validated by reviewing samples submitted for aneuploidy testing (ANP v3) for mention of “heparin” or “lovenox.” There were 26 patients matching these criteria, and they were heavily enriched for test failures (e.g. QC fails or no-call results). Upon review of case notes, 15/26 were confirmed by the patient or clinician to be on lovenox. Eleven patients were confirmed to not be on lovenox. The fetal fraction, estimated genomic equivalents, and fetal genomic equivalents for these samples are summarized in Table 1. As a comparison, the data for 18,112 consecutive samples is also shown.

TABLE 1
Association of heparin use with fetal fraction and fetal DNA molecules.

| | Lovenox, | | No Lovenox, | | Presumed_no, | |
|---|---|---|---|---|---|---|
| Characteristic | N = 15¹ | | N = 11¹ | | N = 18,112¹ | |
| Fetal fraction | 0.04 | (0.02, 0.05) | 0.04 | (0.02, 0.12) | 0.08 | (0.06, 0.11) |
| Gestation age (days) | 105 | (97, 126) | 117 | (104, 131) | 89 | (79, 107) |
| Maternal weight (lbs) | 187 | (134, 229) | 212 | (167, 243) | 163 | (139, 196) |
| Unknown | 5 | | 4 | | 1,863 | |
| Fetal cfDNA | 1,374 | (927, 1,647) | 166 | (83, 262) | 261 | (181, 374) |
| molecules | | | | | | |
| Total cfDNA | 35,656 | (21,454, 43,681) | 2,366 | (1,863, 5,731) | 3,168 | (2,316, 4,417) |
| molecules | | | | | | |
| f*sqrt(g) | 8.07 | (5.31, 10.40) | 3.41 | (1.92, 4.04) | 4.71 | (3.46, 6.35) |
| LOD (ppm) | 84 | (69, 140) | 1,268 | (642, 1,616) | 947 | (679, 1,295) |

¹Median (IQR)

Lovenox use was associated with lower fetal fraction, 4% on average vs 8% in the unselected population. However, the total fetal DNA was increased for the lovenox patients by 5-fold. This is explained by the >10× higher total cfDNA recovered from patients treated with lovenox.

For diagnostic applications that involve quantitative assessments of cell-free DNA (e.g. fetal risk of recessive conditions like sickle cell disease, longitudinal monitoring of circulating tumor DNA), the signal to noise ratio is proportional to the fetal fraction and the square root of the number of total cell-free DNA. The lovenox patients had 1.7-fold higher signal to noise ratio compared to the controls. This is equivalent to 70% higher fetal fraction or 70% lower CV. For detection applications, the limit of detection is 3 or more tissue-specific molecules. Since fetal DNA was increased by 5× in the lovenox treated patients, then the LOD is lowered by a corresponding amount. The LOD could thus be 0.02% for a single locus.

A confounding factor of this analysis is that lovenox-treated patients are enriched for people with autoimmune disorders such as antiphospholipid syndrome and lupus. Lupus is associated with high levels of cfDNA. Thus, some of the observed effect may not be caused by lovenox. However, the effect is so large it is likely that lovenox is acting to increase cfDNA, perhaps by lengthening the cfDNA half-life.

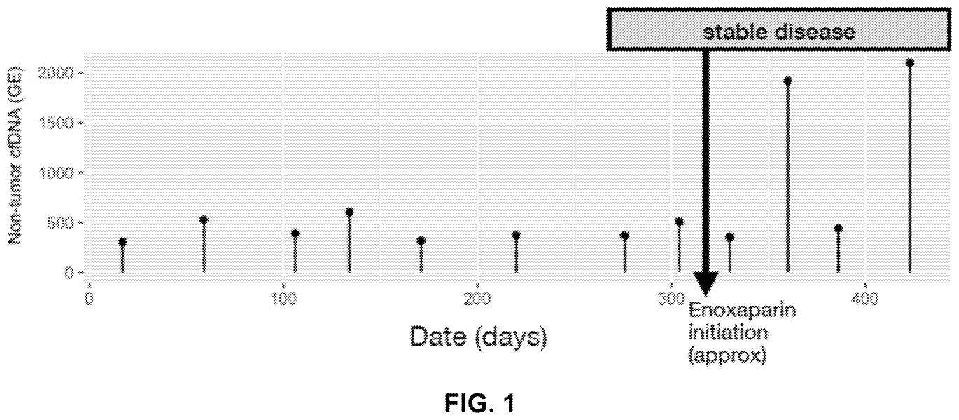

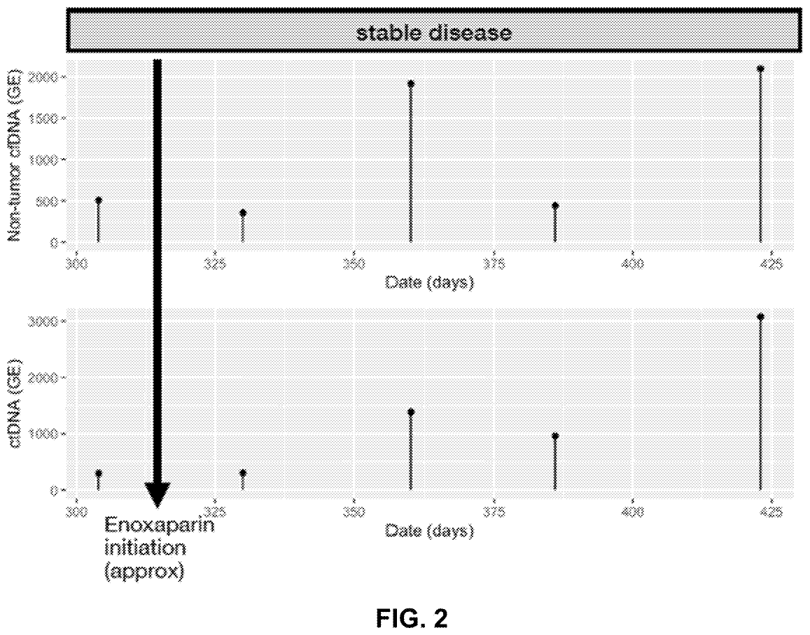

A similar phenomenon is observed for cancer patients where the application of a blood thinner/DNASE1L3 inhibitor agent increases the amount of tumor-originating DNA in the patient's blood. FIGS. 1 and 2 illustrate this with data from longitudinal cell-free DNA monitoring of a metastatic colorectal cancer patient. Twelve blood draws were obtained over the course of ~420 days for the metastatic colorectal cancer patient. Low molecular weight heparin (LMWH; enoxaparin) therapy was initiated at day ~320, as indicated by the arrow.

FIG. 1 illustrates that non-tumor cfDNA increases dramatically after initiation of LMWH. The amount of non-tumor cell-free DNA in this patient prior to LMWH treatment was stable in the preceding 300 days, with a mean and standard deviation of 425 GE and 109 GE, respectively. After initiation of LMWH, there are multiple blood draws that exhibited a ~5× increase in the non-tumor cfDNA, at about 2000 GE.

FIG. 2 illustrates that ctDNA increases alongside non-tumor cell-free DNA. The patient was assessed to have stable disease by clinical and radiographic factors in the time range of day 300 to day 425. For the blood draws exhibiting spikes in non-tumor DNA (on days 360 and 423), the ctDNA is also increased. This demonstrates that both total cell-free DNA and ctDNA is increased after LMWH treatment. Although the effects of LMWH are described, other variants of heparin or heparin derivatives may be used, such as unfractionated heparin (UFH). In some embodiments, the heparin or heparin derivative has a molecular weight of greater than 1700 Da. Other compounds that exhibit DNASE1L3 inhibitory activity may also be used, such as dextran 40 or dextran 70.

Thus, for liquid biopsy assays for which the limit of detection is governed by the total number of relevant molecules present, the application of heparin or other blood thinners prior to conducting a blood draw can increase the sensitivity of the assay by making more relevant molecules (e.g., fetal DNA or tumor-originating DNA) available in the blood sample.

### Diagnostic Application: Heparin as a “Priming” Agent for Liquid Biopsy

Heparin can be used as a “priming” agent in liquid biopsy workflows to increase the number of detectable ctDNA molecules, which is particularly beneficial in early-stage cancer detection or minimal residual disease monitoring where ctDNA levels are extremely low. Even in advanced cancer patients, the level of ctDNA remains low enough that false-negatives are an important concern. Thus, use of heparin as a cfDNA priming agent can also be useful in improving the sensitivity of ctDNA comprehensive genomic profiling assays used for therapy selection in metastatic cancer.

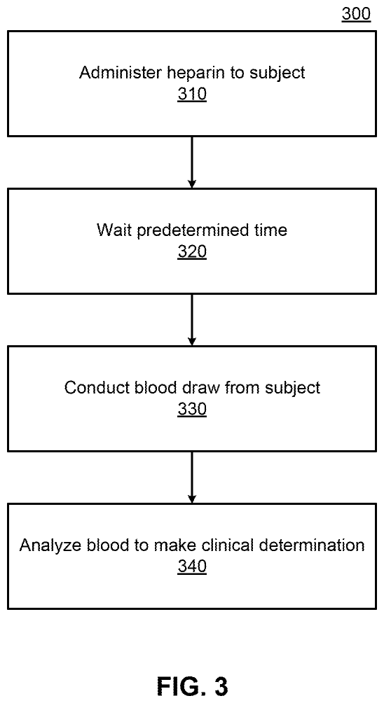

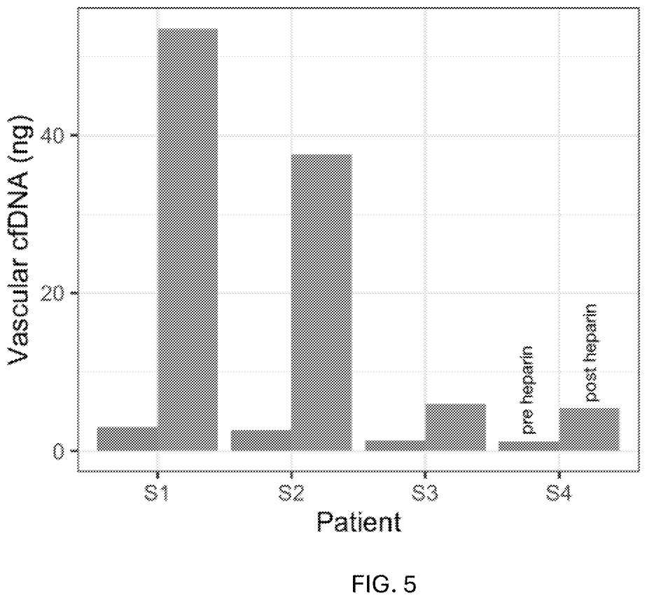

As shown in FIG. 5, pretreatment with heparin increases not just the amount of total cfDNA, but also the amount of cfDNA from vascular endothelial cells (VECs). More than 90% of cfDNA in a sample is from white blood cells, rather than tumors. However, pretreatment with heparin increased both the cfDNA from WBCs and the cfDNA from VECs. Therefore, the increase in cfDNA is not limited to any single tissue and is also increased in ctDNA. FIG. 3 illustrates one embodiment of a clinical diagnostic workflow. In the embodiment shown, the workflow includes administering 310 a controlled dose of heparin (e.g., 5000 U) to a subject and then waiting 320 a predetermined amount of time prior to blood collection for liquid biopsy. In various embodiments, the predetermined amount of time is approximately one to twelve hours or any subject thereof, such as approximately 1-2 hours, approximately 2-3 hours, approximately 3-4 hours, approximately 4-5 hours, approximately 5-6 hours, approximately 2-6 hours, approximately 6-7 hours, approximately 7-8 hours, approximately 8-9 hours, approximately 9-10 hours, approximately 10-11 hours, approximately 11-12 hours, or approximately 12-24 hours. The timing between heparin/heparin-derivative dosage and blood collection depends on the interplay of pharmacokinetics/pharmacodynamics of heparin and cell-free DNA half-life. For example, since the cfDNA half-life is typically ~1 hour, waiting at least 2-4 hours after dosing is ideal so that enough time has elapsed for cfDNA to accumulate in blood at higher levels. On the other hand, heparin and heparin derivatives are cleared with typical half-life of 60-90 minutes. Thus, waiting too long risks collecting blood after the priming effect has already dissipated. However, in some embodiments, it may be desirable to wait 10-12 hours or even 12-24 hours to allow the subject to take the heparin (or other agent that boosts the amount of cfDNA in the subject's blood) the night before a schedule blood draw while still seeing elevated ctDNA levels at the time of the blood draw.

Regardless of the precise timing, the resulting increase in ctDNA molecules enhances the probability of detecting low-abundance tumor-specific genomic alterations. This approach can be particularly valuable in scenarios where ctDNA is at the lower limit of detection of current technologies, thus expanding the clinical utility of liquid biopsy to earlier disease stages or improving the sensitivity of minimal residual disease monitoring.

Once the blood draw has been conducted 330, the blood can be analyzed 340 using any appropriate analysis technique, such as extracting cfDNA, performing library preparation, sequencing the resulting sequencing library, and analyzing the sequencing results to identify the presence or absence (or amount) of sequences associated with a tumor. The library should be sequenced at a depth that allows full sequencing of the increased cfDNA mount, e.g., a sequencing depth of about 10,000× per genomic locus.

### Heparin as a “Priming” Agent for Prenatal Screening

Heparin can also be useful as a priming agent to increase the levels of cfDNA in a blood sample collected from a pregnant woman for screening purpose. Cell-free DNA is a non-invasive method for screening for genomic abnormalities of a fetus. However, amounts of cfDNA can lead to inconclusive results.

Administration of heparin or a heparin derivative prior to drawing the blood for cfDNA analysis can be useful to increase the amount of cfDNA in a sample, and make it less likely to return inconclusive results.

### Validation of Approach

The concentration of cfDNA in circulation represents an equilibrium between generation and clearance processes. Cell-free DNA is primarily released through apoptosis of various cell types, with additional contributions from necrosis and active secretion. The clearance of cfDNA involves multiple mechanisms, with plasma nucleases, particularly DNase1 and DNase1L3, playing central roles in the enzymatic degradation of these DNA fragments. Under normal physiological conditions, cfDNA has a relatively short half-life of approximately 30 minutes to 1 hour, reflecting rapid clearance mechanisms.

Heparin, a widely used anticoagulant in clinical settings, has been shown in vitro to have dual effects on plasma nucleases, inhibiting DNase1 while activating DNase1L3. This creates uncertainty regarding the net effect of heparin on cfDNA metabolism in vivo. Interestingly, previous clinical observations have noted higher test failure rates in NIPT for patients receiving heparin therapy, with one study reporting a 20-fold increase in failure rates associated with heparin use. Therefore, the effects of heparin on cell-free DNA in vivo and the broader implications for cfDNA diagnostics remain poorly understood.

A study was conducted to investigate the effects of heparin administration on cfDNA levels in vivo and explore the potential applications of these findings for enhancing cfDNA diagnostic approaches, particularly in the context of liquid biopsy for cancer detection. The study analyzed two distinct patient cohorts to evaluate the effects of heparin on cfDNA levels:

NIPT Cohort: Pregnant women (n=18138) undergoing routine non-invasive prenatal testing for fetal aneuploidies were evaluated. This cohort included 15 patients receiving therapeutic heparin or low-molecular-weight heparin derivatives for various clinical indications and 18,112 consecutively submitted samples who have no known clinical history of heparin use.

Cancer Patient Case Study: A patient with metastatic colorectal cancer undergoing longitudinal cfDNA monitoring was included as a case study. This patient initiated therapeutic heparin during the monitoring period due to a stable pulmonary embolism, providing an opportunity to assess changes in cfDNA levels before and after heparin treatment initiation.

### Sample Collection and Processing

Blood samples were collected in Streck cell-free DNA tubes and shipped to a centralized diagnostic laboratory. Plasma was separated and either the BillionToOne UNITY Aneuploidy Screen (NIPT Cohort) or the Northstar Response (Cancer Patient Case Study) was performed.

Quantification of Total and Specific cfDNA Fractions

For the NIPT cohort, total cfDNA was quantified using QCT technology, which provided absolute quantification of DNA sequences through a multiplex PCR and next generation sequencing (NGS) workflow. Briefly, synthetic DNA molecules (QCT molecules) are added to the cfDNA sample prior to DNA amplification. The QCT molecules share PCR priming regions with the multiplex PCR amplicons and also incorporate a molecular barcode. Thus, the QCT molecules and cfDNA molecules amplify at the same rate, and individual QCT molecules are identifiable in the NGS sequencing reads through clustering of the molecular barcode.

Fetal fraction in the NIPT cohort was determined by analyzing the cell-free DNA sample for polymorphic SNPs. SNPs that are present at ~1-10% allele fraction are assumed to originate from fetal DNA due to fetal inheritance of a non-maternal allele.

Contrary to expectations based on the known in vitro effects of heparin on nucleases, the study showed significantly higher levels of total cfDNA in pregnant women receiving heparin therapy compared to controls (median: 35,700 haploid genomic equivalents [GE] vs 3,200 GE, p<0.001). Not only was total cfDNA increased, but fetal cfDNA was also highly elevated in the heparin-treated group. The median detected fetal cfDNA molecules was 5.3-fold higher in the heparin group compared to controls (median: 1,374 GE vs. 261 GE, p<0.001). Interestingly, this was despite the fetal fraction being lower in the heparin group relative to control (median 4% fetal fraction vs 8% fetal fraction, p<0.001).

The decreased fetal fraction explains prior reports that heparin use is associated with higher NIPT failure rates, given that QC thresholds are often set by fetal fraction. However, this finding shows that heparin increases the absolute level of fetal-derived cfDNA.

For the cancer patient case study, cell-free DNA was subjected to the Northstar Response Assay. Briefly, a multiplex PCR is performed on bisulfite-converted DNA. Northstar Response incorporates amplicons to measure both total cfDNA and tumor-derived cfDNA (i.e., ctDNA). The total cfDNA is measured by amplicons that have no known CpG methylation differences across all tissues. Additionally, tumor-specific amplicons are included to target CpG methylation that is specific to tumor tissue. QCTs for each amplicon enable absolute quantification of both non-tumor and tumor-specific cfDNA.

The study showed a dramatic increase in total cfDNA levels following the initiation of therapeutic heparin for pulmonary embolism. Prior to heparin treatment, the patient's baseline non-tumor cfDNA concentration was relatively stable across eight blood collections spanning ~300 days (mean 425 GE, std dev 109 GE). Following heparin initiation, cfDNA levels increased nearly 5-fold to ~2000 GE in two independent timepoints. There were also timepoints after enoxaparin initiation that have low cfDNA levels that are typical of the historical average; the variability of cfDNA obtained while on heparin may be due to differences in timing between heparin dosage and blood collection.

Significantly, the absolute quantity of ctDNA also increased substantially following heparin administration. For the two timepoints with increased non-tumor cfDNA, tumor-specific ctDNA also increased, with measurements of 1500 GE and 3000 GE. This increase in ctDNA occurred despite stable disease as assessed by imaging during this period, suggesting that the observed changes were attributable to heparin's effect on cfDNA metabolism rather than to disease progression.

This demonstrates that heparin administration in vivo leads to a significant increase in circulating cfDNA levels, affecting both total cfDNA and specific fractions of interest (fetal cfDNA and tumor-derived cfDNA). These observations were consistent across the NIPT cohort and the cancer patient case study, suggesting a generalizable effect of heparin on cfDNA metabolism.

The in vivo effect of heparin differs from what might be predicted based solely on its in vitro interactions with plasma nucleases. While heparin activates DNase1 and inhibits DNase1L3 in vitro, the net effect in vivo appears to be a reduction in overall cfDNA clearance, leading to higher steady-state levels of circulating DNA fragments. This suggests that either the inhibitory effect on DNAse1L3 predominates in vivo, or that heparin influences additional mechanisms involved in cfDNA clearance beyond direct nuclease activity.

The increased cfDNA levels observed with heparin use may explain the previously reported higher test failure rates in NIPT for patients receiving heparin therapy. These failures might not be due to analytical interference but could instead reflect altered cfDNA composition or fragment size distribution resulting from disrupted nuclease activity.

### Example 1: Increase in cfDNA with Heparin

Five subjects had blood collected in Streck cell-free DNA blood collection tubes immediately before the first heparin dose (“pre heparin”) and 2-6 hours after heparin (“post heparin”). Plasma was isolated from 10 mL of whole blood, and the cell-free DNA was purified from plasma and quantified using fluorometry.

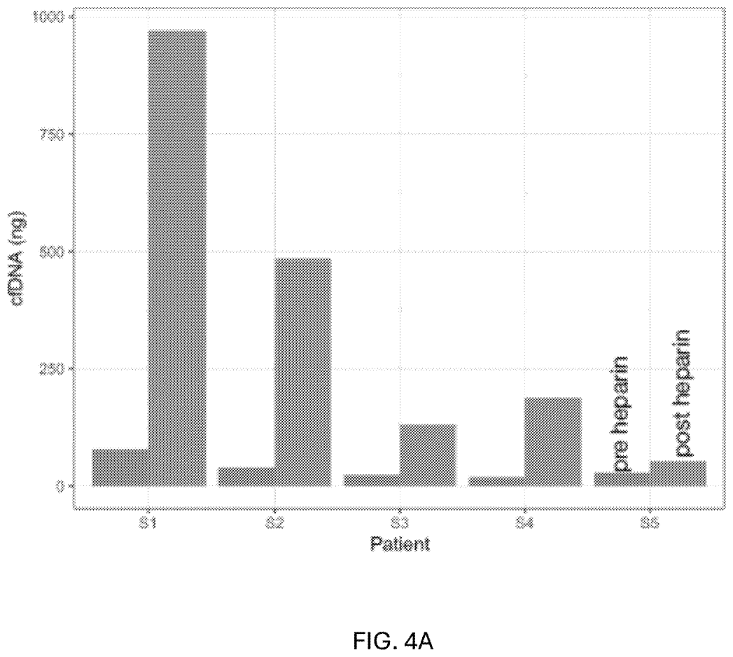

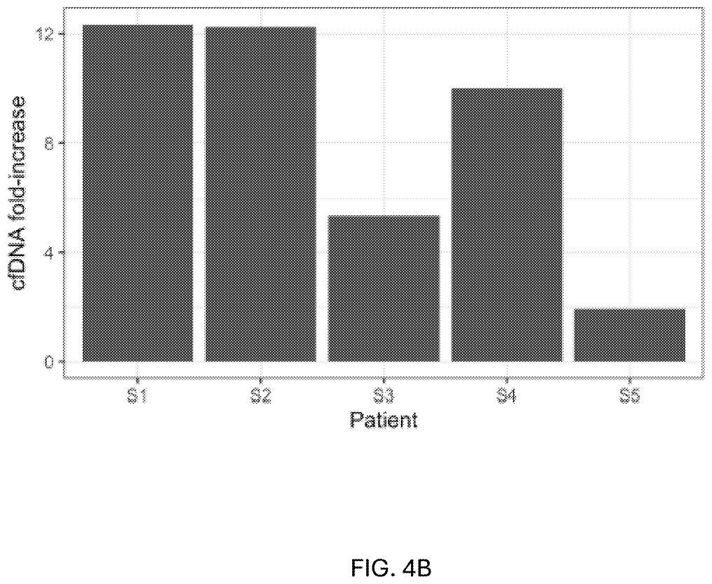

All five subjects had higher total cfDNA after initiating heparin derivatives (e.g. enoxaparin) compared to the baseline blood sample that was collected prior to heparin exposure (FIG. 4A). Subjects S1-S2 also had abdominal surgery in between the pre- and post-heparin time points. Subjects S3-S5 had blood drawn on the same day. The fold-change of cfDNA after heparin exposure ranged from 2× to 12× increase (FIG. 4B). The mean fold change was 8.4×(2.7-14×95% confidence interval; p=0.015).

Greater than 90% of cfDNA ordinarily originates from leukocytes. The total amount of vascular endothelial cell (VEC) DNA was determined by measuring the total cfDNA purified from 10 mL of whole blood and the proportion of cfDNA that derived from VEC. The VEC proportion was measured by performing multiplex PCR and next-generation DNA sequencing (Northstar Response assay) on bisulfite-converted DNA to measure DNA methylation patterns at loci that are specifically hypomethylated in endothelial cells. The total amount of DNA was also measured using internal control loci that are constitutively methylated. The absolute number of methylated or unmethylated DNA molecules present in each locus was measured using quantitative counting templates (QCTs). The fraction of VEC is the count of VEC DNA molecules divided by the count of constitutively methylated molecules measured in the sample.

FIG. 5 shows that the total amount of cfDNA originating from vascular endothelial cells is increased by heparin.

The increase in cfDNA from VEC cells makes this approach particularly useful for measuring tumor fraction, since it increases the proportion of cfDNA in a sample derived from tumors, compared to the proportion derived from WBCs.

### Additional Considerations

Any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment. Similarly, use of “a” or “an” preceding an element or component is done merely for convenience. This description should be understood to mean that one or more of the elements or components are present unless it is obvious that it is meant otherwise.

Where values are described as “approximate” or “substantially” (or their derivatives), such values should be construed as accurate+/−10% unless another meaning is apparent from the context. For example, “approximately ten” should be understood to mean “in a range from nine to eleven.”

The terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Upon reading this disclosure, those of skill in the art will appreciate still additional alternative structural and functional designs. Thus, while particular embodiments and applications have been illustrated and described, it is to be understood that the described subject matter is not limited to the precise construction and components disclosed. The scope of protection should be limited only by any claims that ultimately issue.

What is claimed is:

1. A liquid biopsy assay comprising:
causing administration of an agent to a subject that increases a total amount of cfDNA in the subject's blood;
waiting approximately a predetermined period of time;
obtaining a blood sample from the subject; and
analyzing the blood sample to make a diagnostic determination.

2. The liquid biopsy of claim 1, wherein the agent is a DNASE1L3 inhibitor.

3. The liquid biopsy of claim 2, wherein the DNASE1L3 inhibitor is heparin, a heparin derivative, aspirin, dextran 40, or dextran 70.

4. The liquid biopsy of claim 1, wherein the predetermined period of time is approximately 1-3 hours, approximately 10-12 hours, or approximately 12-24 hours.

5. The liquid biopsy of claim 1, wherein the subject is a pregnant woman, and the diagnostic determination is determining the presence of absence of a genetic condition in a fetus of the pregnant woman.

6. The liquid biopsy of claim 1, wherein the subject is a cancer patient, and the diagnostic determination is detecting the presence of a tumor in the cancer patient or calculating one or more metrics indicating disease progression in the cancer patient.

7. The liquid biopsy of claim 6, wherein the metric indicating disease progression is minimal residual disease status.

8. The liquid biopsy of claim 1, wherein the agent also decreases a fetal-fraction or tumor-originated-fraction of the subject's blood.

9. The liquid biopsy of claim 1, wherein the analyzing comprises:
extracting cell free DNA (cfDNA) from the blood sample;
forming a sequencing library using the cfDNA;
sequencing the sequencing library to generate sequencing data; and
making the diagnostic determination based on the sequencing data.

10. The liquid biopsy of claim 9, wherein the sequencing library is sequenced at a depth of 10,000× per genomic location.

11. A liquid biopsy assay comprising:
obtaining a blood sample, wherein the blood sample was obtained by:
administering an agent to a subject that increases a total amount of cfDNA in the subject's blood;
waiting approximately a predetermined period of time;
conducting a blood draw on the subject to extract the blood sample; and
analyzing the blood sample to make a diagnostic determination.

12. The liquid biopsy of claim 11 wherein the agent is a DNASE1L3 inhibitor.

13. The liquid biopsy of claim 12, wherein the DNASE1L3 inhibitor is heparin, a heparin derivative, aspirin, dextran 40, or dextran 70.

14. The liquid biopsy of claim 11, wherein the predetermined period of time is approximately 1-3 hours, approximately 10-12 hours, or approximately 12-14 hours.

15. The liquid biopsy of claim 11, wherein the subject is a pregnant woman, and the diagnostic determination is determining the presence of absence of a genetic condition in a fetus of the pregnant woman.

16. The liquid biopsy of claim 11, wherein the subject is a cancer patient, and the diagnostic determination is detecting the presence of a tumor in the cancer patient or calculating one or more metrics indicating disease progression in the cancer patient.

17. The liquid biopsy of claim 16, wherein the metric indicating disease progression is minimal residual disease status.

18. The liquid biopsy of claim 11, wherein the agent also decreases a fetal-fraction or tumor-originated-fraction of the subject's blood.

19. The liquid biopsy of claim 11, wherein the analyzing comprises:
extracting cell free DNA (cfDNA) from the blood sample;
forming a sequencing library using the cfDNA;
sequencing the sequencing library to generate sequencing data; and
making the diagnostic determination based on the sequencing data.

20. The liquid biopsy of claim 19, wherein the sequencing library is sequenced at a depth of 10,000× per genomic location.

21. A liquid biopsy assay comprising:
recommending administration of an agent that increases the total amount of cfDNA in a subject's blood to the subject;
obtaining a blood sample from a subject approximately a predetermine period of time after has the agent has been administered to the subject; and
analyzing the blood sample to make a diagnostic determination.

22. The liquid biopsy of claim 21, wherein the agent is a DNASE1L3 inhibitor.

23. The liquid biopsy of claim 22, wherein the DNASE1L3 inhibitor is heparin, a heparin derivative, aspirin, dextran 40, or dextran 70.

24. The liquid biopsy of claim 21, wherein the predetermined period of time is approximately 1-3 hours, approximately 10-12 hours or approximately 12-24 hours.

25. The liquid biopsy of claim 21, wherein the subject is a pregnant woman and the diagnostic determination is determining the presence of absence of a genetic condition in a fetus of the pregnant woman.

26. The liquid biopsy of claim 21, wherein the subject is a cancer patient and the diagnostic determination is detecting the presence of a tumor in the cancer patient or calculating one or more metrics indicating disease progression in the cancer patient.

27. The liquid biopsy of claim 21, wherein the metric indicating disease progression is minimal residual disease status.

28. The liquid biopsy of claim 21, wherein the agent also decreases a fetal-fraction or tumor-originated-fraction of the subject's blood.

29. The liquid biopsy of claim 21, wherein the analyzing comprises:
extracting cell free DNA (cfDNA) from the blood sample;
forming a sequencing library using the cfDNA;
sequencing the sequencing library to generate sequencing data; and
making the diagnostic determination based on the sequencing data.

30. The liquid biopsy of claim 29, wherein the sequencing library is sequenced at a depth of 10,000× per genomic location.
